SEQUENCE LISTING (2 sequences)

SEQ ID NO: 1 - protein, 491 aa, Mus musculus
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 2 - protein, 501 aa, Artificial Sequence
  Nampt-FLAG (tm) protein
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPHGGDYKDDDDK

(19) United States
(12) Patent Application Publication
(10) Pub. No.: US 2011/0123510 A1
(43) Pub. Date: May 26, 2011

(54) Methods of Affecting Biological Function Through Circadian Clock Feedback Cycle by NAMPT-Mediated NAD+ Biosynthesis

(75) Inventors: Shin-ichiro Imai, St. Louis, MO (US); Joseph Bass, Winnetka, IL (US)

(73) Assignee: WASHINGTON UNIVERSITY, St. Louis, MO (US)

(21) Appl. No.: 12/890,035

(22) Filed: Sep. 24, 2010

Related U.S. Application Data
(60) Provisional application No. 61/245,541, filed on Sep. 24, 2009.

(51) Int. Cl. A61K 38/45; A61K 31/455; A61K 31/706; A61K 31/7084; A61P 21/00

(52) U.S. Cl. 424/94.5; 514/355; 514/52; 514/44 R

(57) ABSTRACT

The present invention relates to methods of regulating biological functions in a mammal that are mediated, at least in part, by the circadian clock.

### CROSS REFERENCE TO RELATED APPLICATIONS

This application claims the benefit of U.S. Provisional Application Ser. No. 61/245,541 filed Sep. 24, 2009, the entire content of which is hereby incorporated herein by reference.

### STATEMENT OF GOVERNMENT RIGHTS

This invention was made with Government support. The Government has certain rights in the invention.

### FIELD OF THE INVENTION

The present invention generally relates to processes for regulating the circadian clock and thereby regulating biological functions related to the same in a mammal.

### BACKGROUND

Many aspects of mammalian behavior and physiology are coordinated through interconnected networks of 24 hour central and peripheral oscillators that synchronize cycles of fuel storage and utilization to maintain organismal homeostasis. In mice, circadian disruption has been tied to metabolic disturbance (F. W. Turek et al., Science 308, 1043 (2005); R. D. Rudic et al., PLoS Biol. 2, e377 (2004)), while conversely, high-fat diet alters both behavioral and molecular rhythms (A. Kohsaka et al., Cell Metab. 6, 414 (2007); M. Barnea, Z. Madar, O. Froy, Endocrinology 150, 161 (2009)). The underlying mechanism of the mammalian clock consists of a transcription-translation feedback loop in which CLOCK and BMAL1 activate transcription of Cryptochrome (Cry1 and 2) and Period (Per1, 2, and 3), leading to subsequent repression of CLOCK:BMAL1 by CRY and PER proteins (J. S. Takahashi, H. K. Hong, C. H. Ko, E. L. McDearmon, Nat. Rev. Genet. 9, 764 (2008)). An additional feedback loop involves the transcriptional regulation of Bmal1 by RORα and REV-ERBα (N. Preitner et al., Cell 110, 251 (2002); T. K. Sato et al., Neuron 43, 527 (2004)). Previous studies have also implicated a role for cellular NAD⁺ in the regulation of CLOCK and NPAS2 activity (J. Rutter, M. Reick, L. C. Wu, S. L. McKnight, Science 293, 510 (2001)), an observation consistent with the recent finding that the NAD⁺-dependent protein deacetylase SIRT1 modulates activity of the clock complex (Y. Nakahata et al., Cell 134, 329 (2008); G. Asher et al., Cell 134, 317 (2008)).

### SUMMARY OF THE INVENTION

Among the various aspects of the present invention is the provision of regulating the circadian clock in order to affect biological functions generally regulated by the circadian clock.

Briefly, therefore, the present invention is directed to a method of regulating a biological function in a mammal in need thereof, said function being affected by the circadian clock. The method comprises administering to the mammal a compound selected from nicotinamide or salts or prodrugs thereof, nicotinamide mononucleotide (NMN) or salts or prodrugs thereof, nicotinamide adenine dinucleotide (NAD) or salts or prodrugs thereof, nicotinamide phosphoribosyltransferase (NAMPT), or combinations thereof.

Another aspect of the invention is a method of treating a metabolic disorder in a mammal. The method comprises administering to the mammal a compound selected from nicotinamide or salts or prodrugs thereof, nicotinamide mononucleotide (NMN) or salts or prodrugs thereof, nicotinamide adenine dinucleotide (NAD) or salts or prodrugs thereof, nicotinamide phosphoribosyltransferase (NAMPT), or combinations thereof.

Another aspect of the invention comprises a method of modulating sleep in a mammal in need thereof. The method comprises administering to the mammal a compound selected from nicotinamide or salts or prodrugs thereof, nicotinamide mononucleotide (NMN) or salts or prodrugs thereof, nicotinamide adenine dinucleotide (NAD) or salts or prodrugs thereof, nicotinamide phosphoribosyltransferase (NAMPT), or combinations thereof.

Yet another aspect of the invention comprises a method of treating or preventing a disorder in a mammal mediated by the function of the circadian clock. The method comprises administering to the mammal a compound selected from nicotinamide or salts or prodrugs thereof, nicotinamide mononucleotide (NMN) or salts or prodrugs thereof, nicotinamide adenine dinucleotide (NAD) or salts or prodrugs thereof, nicotinamide phosphoribosyltransferase (NAMPT), or combinations thereof.

Other objects and features will be in part apparent and in part pointed out hereinafter.

### BRIEF DESCRIPTION OF THE DRAWINGS

FIG. 1 illustrates the oscillation of NAMPT-mediated NAD⁺ biosynthesis as demonstrated and further described in Example 1. FIG. 1A graphically illustrates relative expression levels of Nampt RNA in liver and white adipose tissue (WAT) across the 24 hr light:dark cycle. Gray shading indicates dark period (n=4-6 mice/genotype/time point). Figure B is a gel of western blots showing NAMPT and GAPDH at indicated ZTs. Quantitation of NAMPT normalized to GAPDH. FIG. 1C graphically illustrates relative expression levels of Nampt RNA across 48 hrs in liver of WT and Clock^{Δ19} mice in constant darkness. Shading indicates where light and dark periods would normally occur under 12:12 LD conditions. (n=2 WT, n=1 Clock^{Δ19}/time point); (p<0.01, one-way ANOVA for WT oscillation). FIG. 1D graphically illustrates NAD⁺ levels across 48 hrs in liver of WT mice in constant darkness. (n=2 WT/time point). FIG. 1E graphically illustrates NAD⁺ levels in WT and Clock^{Δ19} mutant liver at ZT2 and ZT14 (n=3) (upper panel). Relative expression levels of Nampt RNA (middle panels) and NAD⁺ (lower panels) in Bmal1^{-/-}, Cry1^{-/-}/Cry2^{-/-}, and WT liver at ZT7 (n=4-13). *p<0.05; **p<0.01; ***p<0.001. All data is presented as mean±SEM.

FIG. 2 graphically illustrates NAMPT, NAD⁺, and SIRT1 regulation of CLOCK:BMAL1 activity. FIGS. 2A and 2B graphically illustrate NAD⁺ levels (2A) and relative expression levels of Pepck, G6 Pase, and Igf-1 RNA (2B) in primary hepatocytes incubated with 200 nM FK866 for 24 hours (n=3/group). (Pepck, phosphoenolpyruvate carboxykinase; G6 Pase, glucose-6-phosphatase; Igf-1, insulin-like growth factor-1). FIGS. 2C, 2D, and 2E graphically illustrate relative luciferase activity of Per2-luciferase in the presence of (2C) 10 nM FK866 for 24 or 48 hours, (2D) increasing doses of Sirt1 (200, 400, 800 ng), and (2E) Sirt1 (800 ng) alone or Sirt1 (800 ng) and 10 nM FK866 for 48 hours. *p<0.05; **p<0.01; ***p<0.001; NS not significant. All data is presented as mean±SEM.

FIG. 3 illustrates the NAMPT/NAD⁺-driven feedback loop through SIRT1/CLOCK:BMAL1. FIG. 3A shows gels of ChIP assays in primary hepatocytes for Per2 with rIgG control (left), SIRT1 antibody (middle), and input (right). Primer locations are schematically shown above. FIGS. 3B and 3C graphically illustrate relative expression levels of Per2 RNA in primary hepatocytes following serum shock. Hepatocytes were either incubated with 200 nM FK866 (3B) or infected with SIRT1- and GFP (control)-expressing adenovirus (average of 2 independent experiments) (3C). FIG. 3D shows gels of ChIP assays in liver isolated from mice at CT6 and CT15 for Nampt with no Ab control (left), CLOCK antibody (middle), and input (right). Primer locations are shown schematically above. The black box indicates a canonical E-box, while gray circles represent non-canonical E-boxes. Control primers are located ˜5 kb upstream of the Nampt transcriptional start site. Per2 E-box primers are included as a positive control. FIG. 3E graphically illustrates the relative Nampt expression levels in adenovirally GFP— and Clock/Bmal1-infected mouse embryonic fibroblasts (average of 2 independent experiments).

FIG. 4 is a schematic illustrating a model of NAD⁺ biosynthesis from nicotinamide (Nic) in mammals. (PRPP, 5-phosphoribosyl-1-pyrophosphate; NMN, nicotinamide mononucleotide; NMNAT, nicotinamide mononucleotide adenylyltranferase; NA, nicotinic acid; Trp, tryptophan).

FIG. 5 is a schematic illustrating a model depicting the NAMPT-mediated NAD⁺ biosynthesis pathway and the regulation of CLOCK:BMAL1 by the NAD⁺-dependent SIRT1. Inhibitors (nicotinamide, Ex-527, FK866) and an activator (resveratrol) of the pathway are indicated.

FIG. 6 graphically illustrates alterations in SIRT1 regulation of activity of CLOCK:BMAL1. Relative luciferase activity of Per2-luciferase in the presence of increasing doses of (FIG. 6A) resveratrol (20, 50, and 100 uM), a SIRT1 activator, (FIG. 6B) nicotinamide (1, 5, and 10 mM), a SIRT1 inhibitor at high concentrations due to end-product inhibition, and (Figure C) Ex-527 (0.1 and 1.0 uM), a specific SIRT1 inhibitor. * p<0.05; ** p<0.01; *** p<0.001. All data is presented as mean±SEM.

FIG. 7 is a gel showing the results of SIRT1 co-immunoprecipitates with BMAL1. (Left) Immunoprecipitation with either mouse IgG control or HA and blotting against SIRT1. (Right) Immunoprecipitation with either rabbit IgG control or SIRT1 and blotting against HA.

FIG. 8 illustrates the canonical and non-canonical E-boxes located in the upstream promoter region and first intron of Nampt. Depicted in the solid rectangular box is one canonical E-box (CACGTG) with four flanking nucleotides on either side. Depicted in the dashed rectangular boxes are non-canonical E-boxes.

FIG. 9 is a schematic illustrating a model of NAMPT/NAD⁺-driven feedback loop through SIRT1/CLOCK:BMAL1.

### DETAILED DESCRIPTION OF THE INVENTION

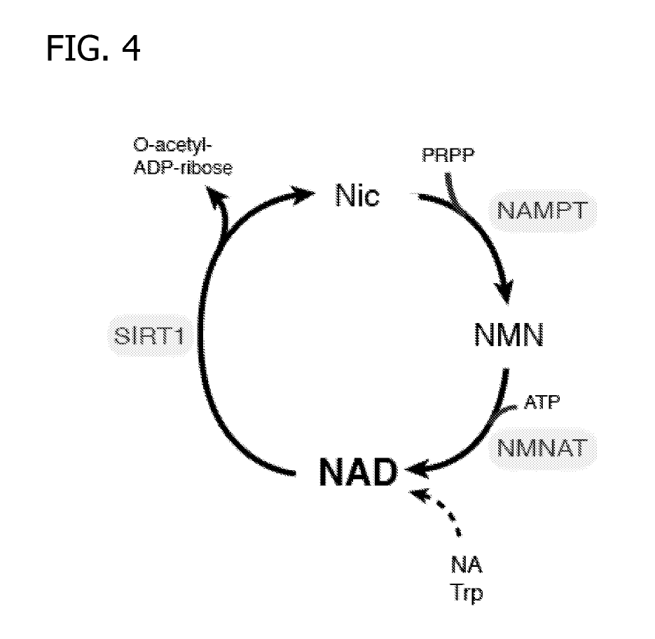

NAD⁺ is a classic co-enzyme that is synthesized from three major precursors—tryptophan, nicotinic acid, and nicotinamide (FIG. 4) (U.S. Patent Application Publication No. US2007/0082373 A1 and U.S. application Ser. No. 11/575,605). In yeast, it has been reported that enzymes involved in the NAD⁺ salvage pathway play an important role in the regulation of Sir2 activity (R. M. Anderson et al., J. Biol. Chem. 277, 18881 (2002); R. M. Anderson, K. J. Bitterman, J. G. Wood, O. Medvedik, D. A. Sinclair, Nature 423, 181 (2003)). On the other hand, in mammals, the predominant NAD⁺ biosynthetic pathway involves the conversion of nicotinamide and 5′-phosphoribosyl-pyrophosphate to nicotinamide mononucleotide (NMN) by the rate-limiting enzyme nicotinamide phosphoribosyltransferase (NAMPT) (FIG. 4) (J. R. Revollo, A. A. Grimm, S. Imai, J. Biol. Chem. 279, 50754 (2004); J. R. Revollo et al., Cell Metab. 6, 363 (2007); S. Imai, Curr. Pharm. Des. 15, 20 (2009)). NAMPT-mediated NAD⁺ biosynthesis has been demonstrated to play a critical role in a number of biological processes through the NAD⁺-dependent deacetylase SIRT1 (FIG. 4) (J. R. Revollo, A. A. Grimm, S. Imai, J. Biol. Chem. 279, 50754 (2004); E. van der Veer et al., J. Biol. Chem. 282, 10841 (2007); M. Fulco et al., Dev. Cell 14, 661 (2008); H. Yang et al., Cell 130, 1095 (2007)).

The circadian clock is encoded by a transcription-translation feedback loop that synchronizes behavior and metabolism with the light-dark cycle. It has been unexpectedly discovered that both the rate-limiting enzyme in mammalian NAD⁺ biosynthesis, nicotinamide phosphoribosyltransferase (NAMPT), and levels of NAD⁺, display circadian oscillations which are regulated by the core clock machinery in mice. Inhibition of NAMPT promotes oscillation of the clock gene Per2 by releasing CLOCK:BMAL1 from suppression by SIRT1. In turn, the circadian transcription factor CLOCK binds to and up-regulates Nampt, thus completing a feedback loop involving NAMPT/NAD⁺ and SIRT1/CLOCK:BMAL1.

Thus, the periodic variation in NAMPT-mediated NAD⁺ biosynthesis suggests that it impacts physiologic cycles and possibly the sleep-wake and fasting-feeding cycle. Without being bound by a single theory, it is believed that NAD⁺ serves as a critical “metabolic oscillator” for the rhythmic regulation of response to environmental cues through control of SIRT1 activity. Thus, identified herein is a new circadian feedback loop through NAMPT-mediated NAD⁺ biosynthesis and a pathway underlying the temporal coupling of metabolic, physiologic, and circadian cycles in mammals.

The recognition of a regulatory pathway involving NAMPT/NAD⁺-SIRT1/CLOCK:BMAL1 has broad implications for understanding how physiologic and behavioral cycles are coordinated with the environmental light-dark cycle. For instance, during sleep, when animals are normally quiescent and fasting, the levels of NAMPT steadily increase, peaking at the beginning of the wakefulness period and coinciding with feeding. As a result of the increase in NAMPT, NAD⁺ rises to stimulate SIRT1, which orchestrates an appropriate metabolic response in liver involving a switch from catabolic to anabolic pathways.

In accordance with this discovery, the present invention provides methods for regulation of the core clock machinery (sometimes also referred to as the circadian clock) of a mammal, thereby affecting behaviors, activities, and/or biological functions that occur in or are affected by a diurnal or circadian cycle and that are regulated, at least in part, by the circadian clock. Generally, the methods involve the administration of a therapeutic or prophylactic amount of a circadian clock-regulating compound to a patient or mammal in need of regulation of the circadian clock.

Methods of Treatment or Prophylaxis

The methods of treatment disclosed herein are generally directed to methods of regulating the circadian clock, thereby regulating or affecting biological functions that are regulated by (sometimes also said to be affected by, affiliated with, or mediated by) the activity of the circadian clock. Typically, these biological functions display a pattern of activity and inactivity that is generally repeated approximately every 24 hours, oscillating between “active” and “inactive” states during the 24 hour period.

Thus, the present invention provides for a method of regulating the activity of the circadian clock by administering to a mammal in need thereof a circadian-clock regulating compound. Generally, the regulation of the activity of the circadian clock is the result of the regulation of CLOCK:BMAL1, which is achieved according to the present methods by regulating the activity of SIRT1. The activity of SIRT1 is generally regulated according to the present methods by administration of a circadian clock-regulating compound, and in certain embodiments, by administration of a compound that affects the NAD⁺ pathway. The regulation of the circadian clock thereby permits regulation of activities mediated by the circadian clock.

According to the present invention, the activity of the circadian clock may be increased, decreased, or maintained by the administration of a circadian clock-regulating compound. Accordingly, biological functions (sometimes also referred to as biological activities) that are regulated by the activity of the circadian clock, may also be increased, decreased, or maintained. In addition, these biological functions may also be time shifted; that is to say, an activity that typically occurs during a particular period, such as for example, during daytime or daylight hours (sometimes also referred to as the light cycle) or during the night or nighttime hours (sometimes also referred to as the dark cycle) may be shifted such that the activity occurs during the dark or light cycle, respectively, instead.

Any of a number of biological functions that are typically affected by the activity of the circadian clock may be regulated by the methods of the present invention. Thus, the present methods may be used to treat disorders or disease states that are the result of, for example, the irregular, inadequate, or pathological function of the circadian clock. Similarly, the present methods may be used to treat disorders or symptomatology caused by exogenous factors that affect the proper function or activity of the circadian clock or that require a “resetting” of the clock. The term “treat,” “treating,” or “treatment” as used herein includes achieving a therapeutic benefit. By therapeutic benefit is meant eradication, amelioration, or prevention of the underlying disorder being treated. For example, in a patient suffering from a metabolic disorder, a therapeutic benefit includes eradication or amelioration of the underlying metabolic disorder. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of NMN to a patient experiencing a metabolic disorder provides therapeutic benefit not only when the patient's serum NMN or NAD level is increased, but also when an improvement is observed in the patient with respect to other disorders that accompany the metabolic disorder, like weight loss or gain. In some treatment regimens, the circadian clock-regulating compound of the invention may be administered to a patient at risk of developing a disorder as described herein or to a patient reporting one or more of the physiological symptoms of such a disorder, even though a diagnosis of a metabolic disorder may not have been made.

Examples disorders, disease states, or symptomatology that may be treated according to the methods of the present invention include, but are not limited to, travel to or across one or more time zones, a change in work shifts, night shift work, or a change in the physical status of a mammal caused by, for example, pregnancy or administration of medications of any kind. Accordingly, the methods of the present invention may be used to treat or prevent disorders, symptoms of disorders, or symptoms caused by exogenous factors. Such disorders and symptoms may include, for example, metabolic disorders, such as improper cycling or timing of feeding and fasting cycles, hyperglycemia, hypoglycemia, or diabetes; sleep disorders, such as insomnia, advanced sleep phase syndrome, delayed sleep phase syndrome, inconsistent sleep/wake cycles, or narcolepsy or to improve wakefulness in individuals suffering from excessive sleepiness; and symptoms caused by exogenous factors, such as, travel to or across one or more time zones (jet lag), shifting into or out of daylight savings time, a change in work shifts or night shift work, pregnancy, or medications being taken for unrelated diseases or disorders.

Accordingly, in one embodiment, the present invention is directed to a method of regulating a biological function in a mammal, the function being affected by the circadian clock. The method comprises administering a therapeutic or prophylactic (sometimes also referred to as a circadian clock-regulating) amount of a circadian clock-regulating compound to the mammal. The biological function can be, for example, any one of the biological functions described herein. In another embodiment, the invention comprises a method of treating a metabolic disorder in a mammal and comprises administering a therapeutic or prophylactic amount of a circadian clock-regulating compound to the mammal. In yet another embodiment, the invention comprises a method of treating a disorder in a mammal mediated by the function of the circadian clock and comprises administering a therapeutic or prophylactic amount of a circadian clock-regulating compound to the mammal. According to any one of these embodiments, the circadian clock-regulating compound may be, for example, nicotinamide, nicotinamide mononucleotide (NMN), nicotinamide adenine dinucleotide (NAD); salts and prodrugs thereof; nicotinamide phosphoribosyltransferase (NAMPT); and combinations thereof, as described in greater detail below. In another embodiment, the circadian clock-regulating compound may be an antagonist of any one of the compounds listed above, thereby exacting an effect opposite that of nicotinamide, nicotinamide mononucleotide (NMN), nicotinamide adenine dinucleotide (NAD); salts and prodrugs thereof; nicotinamide phosphoribosyltransferase (NAMPT); and combinations thereof.

In a particular embodiment, the present invention is directed to a method of regulating metabolic activity of a mammal comprising administering to the mammal a therapeutic amount of a circadian clock-regulating compound. In one embodiment, the metabolic activity of the mammal is increased. In another embodiment, the metabolic activity is decreased. In yet another embodiment, the metabolic activity of the mammal is maintained at a desired level, thereby preventing fluctuations in activity/inactivity. In still another embodiment, the metabolic activity is caused to occur in the light cycle (as opposed to its typical occurrence in the dark cycle). In another embodiment, the metabolic activity is caused to occur in the dark cycle (as opposed to its typical occurrence in the light cycle). In a particular embodiment, the circadian clock-regulating compound is administered to the mammal in order to increase the anabolic activity of the liver (e.g., increase the activity of the metabolic pathways of the liver or shift or switch liver activity from catabolism to anabolism). In another embodiment, the circadian clock-regulating compound is administered to the mammal in order to increase the catabolic activity of the liver (e.g., decrease the activity of the metabolic pathways of the liver or shift or switch liver activity from anabolism to catabolism).

Compounds for Circadian Clock Feedback Regulation

As noted above, compounds that may be utilized for the methods disclosed herein (sometimes referred to as circadian clock-regulating compounds) include nicotinamide, nicotinamide mononucleotide (NMN), nicotinamide adenine dinucleotide (NAD); salts, derivatives, and prodrugs thereof; a purified polypeptide useful in NAD biosynthesis; and combinations thereof.

Nicotinamide, which corresponds to Formula (1),

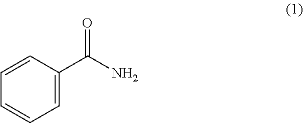

is one of the two principal forms of the B-complex vitamin niacin. The other principal form of niacin is nicotinic acid; nicotinamide, rather than nicotinic acid, however, is the major substrate for nicotinamide adenine dinucleotide (NAD) biosynthesis in mammals, as discussed in detail herein. Nicotinamide, in addition to being known as niacinamide, is also known as 3-pyridinecarboxamide, pyridine-3-carboxamide, nicotinic acid amide, vitamin B3, and vitamin PP. Nicotinamide has a molecular formula of C₆H₆N₂O and its molecular weight is 122.13 daltons. Nicotinamide is commercially available from a variety of sources. The circadian clock-regulating compound may also be a pharmaceutically acceptable salt, derivative, or prodrug of nicotinamide, or combinations thereof.

Nicotinamide mononucleotide (NMN), which corresponds to Formula (2),

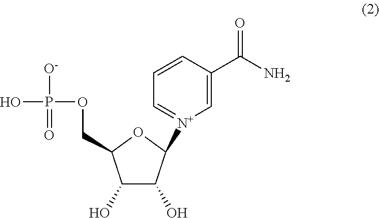

is produced from nicotinamide in the NAD biosynthesis pathway, a reaction that is catalyzed by Nampt. NMN is further converted to NAD in the NAD biosynthesis pathway, a reaction that is catalyzed by Nmnat. Nicotinamide mononucleotide (NMN) has a molecular formula of C₁₁H₁₅N₂O₈P and a molecular weight of 334.22. Nicotinamide mononucleotide (NMN) is commercially available from such sources as Sigma-Aldrich (St. Louis, Mo.). The circadian clock-regulating compound may also be a pharmaceutically acceptable salt, derivative, or prodrug of nicotinamide mononucleotide (NMN), or combinations thereof.

Nicotinamide adenine dinucleotide (NAD), which corresponds to Formula (3):

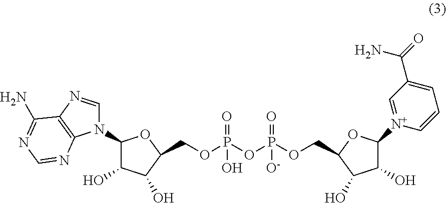

is produced from the conversion of nicotinamide to NMN, which is catalyzed by Nampt, and the subsequent conversion of NMN to NAD, which is catalyzed by Nmnat. Nicotinamide adenine dinucleotide (NAD) has a molecular formula of C₂₁H₂₇N₇O₁₄P₂ and a molecular weight of 663.43. Nicotinamide adenine dinucleotide (NAD) is commercially available from such sources as Sigma-Aldrich (St. Louis, Mo.). The circadian clock-regulating compound may also be a pharmaceutically acceptable salt, derivative, or prodrug of nicotinamide adenine dinucleotide (NAD), or combinations thereof.

Additionally or alternatively, compounds useful in the processes disclosed herein may be a purified polypeptide useful in NAD biosynthesis. In a particular embodiment, the purified polypeptide corresponds to Nampt; in another embodiment, the purified polypeptide corresponds to an extracellular version of Nampt. As discussed above, Nampt catalyses the conversion of nicotinamide to NMN and has been identified as the rate-limiting compound in NAD biosynthesis. Accordingly, administration of a purified polypeptide corresponding to an extracellular version of Nampt in conjunction with the circadian clock-regulating compounds described above may be particularly advantageous. In a particular embodiment, the Nampt has a polypeptide sequence selected from the group consisting of SEQ ID NO: 1; SEQ ID NO: 7; a polypeptide capable of catalyzing the conversion of nicotinamide to nicotinamide mononucleotide (NMN), an amino acid sequence of the polypeptide comprising the amino acid sequence of SEQ ID NO: 1; a polypeptide capable of catalyzing the conversion of nicotinamide to nicotinamide mononucleotide (NMN), an amino acid sequence of the polypeptide comprising the amino acid sequence of SEQ ID NO: 7; a polypeptide capable of catalyzing the conversion of nicotinamide to nicotinamide mononucleotide (NMN), the polypeptide having an amino acid sequence with at least about 65% homology to SEQ ID NO: 1 and conservative amino acid substitutions; and a polypeptide capable of catalyzing the conversion of nicotinamide to nicotinamide mononucleotide (NMN), the polypeptide having an amino acid sequence with at least about 65% homology to SEQ ID NO: 7 and conservative amino acid substitutions.

As used herein, “percent homology” of two amino acid sequences or of two nucleic acids is determined using the algorithm of Karlin and Altschul (Proc. Natl. Acad. Sci. USA, (1990) 87, 2264-2268), modified as in Karlin and Altschul (Proc. Natl. Acad. Sci. USA, (1993) 90, 5873-5877). Such an algorithm is incorporated into NBLAST and XBLAST programs of Altschul et al. (J. Mol. Biol. 215:403-410, 1990). BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res., (1997) 25, 3389-3402). When utilizing BLAST and Gapped BLAST programs, the default parameters of the respective programs (e.g., XBLAST and NBLAST) are used. See, for example, www.ncbi.nlm.nih.gov.

Also included in the scope of circadian clock-regulating compounds are polypeptide analogs of NAMPT arrived at by amino acid substitutions based on the relative similarity of the amino acid side-chain substituents, for example, their hydrophobicity, hydrophilicity, charge, size, etc. One factor that can be considered in making amino acid substitutions is the hydropathic index of amino acids. The importance of the hydropathic amino acid index in conferring interactive biological function on a protein has been discussed by Kyte and Doolittle (J. Mol. Biol., 157: 105-132, 1982). It is accepted that the relative hydropathic character of amino acids contributes to the secondary structure of the resultant protein. This, in turn, affects the interaction of the protein with molecules such as enzymes, substrates, receptors, DNA, antibodies, antigens, etc.

Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant protein having similar biological activity, i.e., still retaining correct biological function. In making such changes, amino acids having hydropathic indices within ±2 are preferably substituted for one another, those within ±1 are more preferred, and those within ±0.5 are most preferred.

Furthermore, amino acid substitutions in the peptides of the present invention can be based on factors other than hydrophobicity, such as size, side chain substituents, charge, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. It should be noted that changes which are not expected to be advantageous can also be useful if these result in the production of functional sequences.

Although the polypeptide may have a polypeptide sequence with at least about 65% homology to SEQ ID NO: 1, in certain embodiments the purified polypeptide has at least about 70% homology to SEQ ID NO: 1. In a particular embodiment, the purified polypeptide has at least about 75% homology to SEQ ID NO: 1; in this embodiment, the purified polypeptide may have, for example, at least about 80%, at least about 85%, at least about 90%, at least about 95%, at least about 97%, or at least about 99% homology to SEQ ID NO: 1.

Although the polypeptide may also have a polypeptide sequence with at least about 65% homology to SEQ ID NO: 7, in certain embodiments the purified polypeptide has at least about 70% homology to SEQ ID NO: 7. In a particular embodiment, the purified polypeptide has at least about 75% homology to SEQ ID NO: 7; in this embodiment, the purified polypeptide may have, for example, at least about 80%, at least about 85%, at least about 90%, at least about 95%, at least about 97%, or at least about 99% homology to SEQ ID NO: 7.

Any one (or a combination) of the compounds disclosed herein may be employed. For example, nicotinamide and a purified polypeptide useful in NAD biosynthesis (such as intracellular or extracellular Nampt) may be administered together or in succession. Alternatively, nicotinamide mononucleotide (NMN) may be administered alone. Where the mammal has sufficient (or elevated) levels of nicotinamide in the blood, a purified polypeptide useful in NAD biosynthesis (such as extracellular Nampt) may also be administered alone.

As noted above, the compound may be a salt of nicotinamide, nicotinamide mononucleotide (NMN), or nicotinamide adenine dinucleotide (NAD). Typically, the salt will be a pharmaceutically acceptable salt; that is, a salt prepared from pharmaceutically acceptable non-toxic acids, including inorganic acids and organic acids. Suitable non-toxic acids include inorganic and organic acids of basic residues such as amines, for example, acetic, benzenesulfonic, benzoic, amphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, barbaric acid, p-toluenesulfonic and the like; and alkali or organic salts of acidic residues such as carboxylic acids, for example, alkali and alkaline earth metal salts derived from the following bases: sodium hydride, sodium hydroxide, potassium hydroxide, calcium hydroxide, aluminum hydroxide, lithium hydroxide, magnesium hydroxide, zinc hydroxide, ammonia, trimethylammonia, triethylammonia, ethylenediamine, lysine, arginine, ornithine, choline, N,N″-dibenzylethylenediamine, chloroprocaine, diethanolamine, procaine, n-benzylphenethylamine, diethylamine, piperazine, tris(hydroxymethyl)-aminomethane, tetramethylammonium hydroxide, and the like. Pharmaceutically acceptable salts of the circadian clock-regulating compounds can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Since prodrugs are known to enhance numerous desirable pharmaceuticals (e.g., solubility, bioavailability, manufacturing), the circadian clock-regulating compound may be delivered in prodrug form. Thus, the present invention is intended to cover the prodrugs of circadian clock-regulating compounds, methods of delivering the same and compositions containing them. “Prodrugs” include any covalently bonded carriers which release an active parent drug in vivo when such prodrug is administered to a mammalian subject. Prodrugs are generally prepared by modifying functional groups present in the compound in such a way that the modifications are cleaved, either in routine manipulation or in vivo, to the parent compound. Prodrugs include compounds of the present invention wherein a hydroxyl or amino group is bonded to any group that, when the prodrug is administered to a mammalian subject, cleaves to form a free hydroxyl or free amino group, respectively. Examples of prodrugs include, but are not limited to, acetate, formate, and benzoate derivatives of alcohol and amine functional groups in the compounds and conjugates of the present invention. Prodrugs of the circadian clock-regulating compound are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. Prodrugs may refer to compounds that are rapidly transformed in vivo to yield the circadian clock-regulating compound, for example by hydrolysis in blood. A thorough discussion of prodrugs is provided in the following: Design of Prodrugs, H. Bundgaard, ea., Elsevier, 1985; Methods in Enzymology, K. Widder et al, Ed., Academic Press, 42, p. 309-396, 25 1985; A Textbook of Drug Design and Development, Krogsgaard-Larsen and H. Bundgaard, ea., Chapter 5; “Design and Applications of Prodrugs” p. 113-191, 1991; Advanced Drug Delivery Reviews, H. Bundgard, 8, p. 1-38, 1992; Journal of Pharmaceutical Sciences, 77, p. 285, 30 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ea., American Pharmaceutical Association and Pergamon Press, 1987, which are incorporated herein by reference.

Dosage/Amount of the Circadian Clock-Regulating Compound and Time Course of Treatment

The dose or amount of the circadian clock-regulating compound administered to the mammal should be an effective amount for the intended purpose, i.e., affecting the circadian clock and/or biological functions related thereto as described herein. Generally speaking, the effective amount of the circadian clock-regulating compound administered to the mammal can vary according to a variety of factors such as, for example, the age, weight, sex, diet, route of administration, and the medical condition of the mammal. Specifically preferred doses are discussed more fully below. It will be understood, however, that the total daily usage of the compounds described herein will be decided by the attending physician or veterinarian within the scope of sound medical judgment.

The specific therapeutically effective dose level for any particular mammal will depend upon a variety of factors including the disorder being treated and the severity of the disorder; activity of the specific compound(s) employed; the age, body weight, general health, sex and diet of the patient; the time of administration; the route of administration; the rate of excretion of the specific compound(s) employed; the duration of the treatment; drugs used in combination or coincidental with the specific compound(s) employed and like factors well known in the medical and/or veterinary arts. For example, it is well within the skill of the art to start doses of the compound(s) at levels lower than those required to achieve the desired therapeutic effect and to gradually increase the dosage until the desired effect is achieved. If desired, the effective daily doses may be divided into multiple doses for purposes of administration. Consequently, single dose compositions may contain such amounts or submultiples to make up the daily dose.

Administration of the circadian clock-regulating compound(s) can occur as a single event or over a time course of treatment. For example, one or more of the circadian clock-regulating compounds can be administered daily, weekly, bi-weekly, or monthly. For treatment of acute conditions, the time course of treatment may be at least several hours or days. Certain conditions could extend treatment from several days to several weeks. For example, treatment could extend over one week, two weeks, or three weeks. For more chronic conditions, treatment could extend from several weeks to several months, a year or more, or the lifetime of the mammal in need of such treatment. Alternatively, the circadian clock-regulating compounds can be administered daily, weekly, bi-weekly, or monthly, for a period of several weeks, months, years, or over the lifetime of the mammal as a prophylactic measure.

Typically, the circadian clock-regulating effective amount is at least about 10 mg/kg of a compound selected from the group consisting of nicotinamide, nicotinamide mononucleotide (NMN), and nicotinamide adenine dinucleotide (NAD); salts, derivatives, or prodrugs thereof; a purified polypeptide useful in NAD biosynthesis (such as, for example, one or more forms of Nampt); and combinations thereof. For example, the circadian clock-regulating amount may be from about 10 mg/kg to about 2,000 mg/kg; from about 100 mg/kg to about 1,900 mg/kg; from about 200 mg/kg to about 1,800 mg/kg; from about 300 mg/kg to about 1,700 mg/kg; from about 400 mg/kg to about 1,600 mg/kg; or from about 500 mg/kg to about 1,500 mg/kg. By way of another example, the circadian clock-regulating amount may be from about 10 mg/kg to about 1,000 mg/kg; from about 20 mg/kg to about 900 mg/kg; from about 30 mg/kg to about 900 mg/kg; from about 40 mg/kg to about 800 mg/kg; or from about 50 mg/kg to about 500 mg/kg. These effective amounts of circadian clock-regulating compounds may be daily, weekly, bi-weekly, or monthly doses. Preferably, the effective amounts are administered as a daily dose.

Routes of Administration, Formulations/Pharmaceutical Compositions

Circadian clock-regulating compounds useful in the present invention may be dispersed in a pharmaceutically acceptable carrier prior to administration to the mammal. The carrier, also known in the art as an excipient, vehicle, auxiliary, adjuvant, or diluent, is typically a substance which is pharmaceutically inert, confers a suitable consistency or form to the composition, and does not diminish the efficacy of the circadian clock-regulating compound. The carrier is generally considered to be “pharmaceutically or pharmacologically acceptable” if it does not produce an unacceptably adverse, allergic or other untoward reaction when administered to a mammal, especially a human.

The selection of a pharmaceutically acceptable carrier will also, in part, be a function of the route of administration. In general, the circadian clock-regulating compounds of the present invention can be formulated for any route of administration so long as the blood circulation system is available via that route. For example, suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration. Preferably, the compounds are formulated for oral dosage and preferably in the form of tablets, pills, dragees, capsules, liquids, gels, syrups, slurries, suspensions, wafers, and the like, for oral ingestion by a patient to be treated. In one embodiment, the oral composition does not have an enteric coating. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose or sucrose; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP); and various flavoring agents known in the art. If desired, disintegrating agents may be added, such as the cross-linked polyvinyl pyrrolidone, agar, or alginic acid or a salt thereof such as sodium alginate.

Pharmaceutically acceptable carriers for use in the circadian clock-regulating compounds of the present invention are well known to those of ordinary skill in the art and are selected based upon a number of factors: the particular compound used, and its concentration, stability and intended bioavailability; the subject, its age, size and general condition; and the route of administration. Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2-30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-((β-hydroxyethyl)-lactamide, N,N-dimethylacetamide_amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di, or tri-glycerides, fatty acid esters such as isopropyl myristrate, fatty acid derived PEG esters such as PEG-hydroxyoleate and PEG-hydroxystearate, N-methylpyrrolidinone, pluronic 60, polyoxyethylene sorbitol oleic polyesters such as poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄—C₂₂ fatty acid(s) (e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2-30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3-30 carbon atoms (e.g., acetone, methyl ethyl ketone, methyl isobutyl ketone); aliphatic, cycloaliphatic or aromatic hydrocarbons having 4-30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1-30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.)(Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.)(Marcel Dekker, Inc., New York, N.Y., 1980), Remington's Pharmaceutical Sciences (A. Gennaro, ed., 19th ed.)(Mack Publishing, Easton, Pa., 1995), The United States Pharmacopeia 24, The National Formulary 19, (National Publishing, Philadelphia, Pa., 2000), A. J. Spiegel et al., and Use of Nonaqueous Solvents in Parenteral Products, JOURNAL OF PHARMACEUTICAL SCIENCES, Vol. 52, No. 10, pp. 917-927 (1963).

Applicant makes no statement, inferred or direct, regarding the status of any of the references cited throughout this application as prior art. All references cited herein are referenced in their entirety and are hereby incorporated herein by reference in their entirety.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

All animal care and use procedures were in accordance with guidelines of the Institutional Animal Care and Use Committee. Clock^{Δ19} (M. H. Vitaterna et al., Science 264, 719 (1994)), Bmal1^{-/-} (M. K. Bunger et al., Cell 103, 1009 (2000)) and Cry1^{-/-}/Cry2^{-/-} (M. H. Vitaterna et al., Proc. Natl. Acad. Sci. USA 96, 12114 (1999)) mutant mice and littermate controls were maintained on a 12:12 LD cycle in the Northwestern University Center for Comparative Medicine. For the constant darkness experiments, male C57BL/6J mice of 6-8 weeks of age were purchased from the Jackson Laboratory and maintained for two weeks on a 12:12 LD cycle. After placement in constant darkness (DD), two male mice were sacrificed every four hours, beginning at 36 hours after their start of DD for two complete 24 hr cycles. Mice were sacrificed by cervical dislocation, and the optic nerves were removed in complete darkness using an infrared viewer (Night Vision D-2MV Goggle/Binocular).

### Example 1

Quantitative Real-Time PCR. Total RNA was extracted from frozen tissue with Tri Reagent (Molecular Research Center, Inc.). cDNA was synthesized using the High Capacity cDNA Reverse Transcription kit (Applied Biosystems). Real-time PCR was performed and analyzed using an Applied Biosystems 7900HT Fast Real-Time PCR System (Applied Biosystems). PCR conditions were: 10 min at 95° C., then 35 cycles of 10 s at 95° C., 15 s at 60° C. Relative expression levels (normalized to Gapdh) were determined using the comparative CT method.

Western Blotting. Protein was extracted from frozen tissue using CelLytic™ MT Mammalian Tissue Lysis Reagent (Sigma Aldrich) supplemented with protease inhibitors. 30 μg of tissue extracts were analyzed by Western blotting with an α-NAMPT (J. R. Revollo, A. A. Grimm, S. Imai, J. Biol. Chem. 279, 50754 (2004)) and α-GAPDH antibody (Calbiochem). Results were quantitated using LabWorks Image Acquisition and Analysis Software (UVP BioImaging Systems).

NAD⁺Measurements. NAD⁺ levels were measured using HPLC with Waters 515 pumps and a 2487 detector with a Supelco LC-18-T column (15 cm×4.6 cm). Briefly, frozen liver tissues or cultured cells were extracted in 1M perchloric acid and neutralized in 3M K₂CO₃ on ice. After centrifugation, the supernatant was mixed with Buffer A (50 mM K₂PO₄/KHPO₄, pH 7.0) and loaded on to the column. The HPLC was run at a flow rate of 1 ml/min with 100% Buffer A from 0-5 min, a linear gradient to 95% Buffer N5% Buffer B (100% methanol) from 5-6 min, 95% Buffer N5% Buffer B from 6-11 min, a linear gradient to 85% Buffer N15% Buffer B from 11-13 min, 85% Buffer A/15% Buffer B from 13-23 min, and a linear gradient to 100% Buffer A from 23-24 min. NAD⁺ eluted as a sharp peak at 15 min and was quantitated based on the peak area compared to a standard curve and normalized to tissue weight of frozen liver tissues or to protein content of cultured cells.

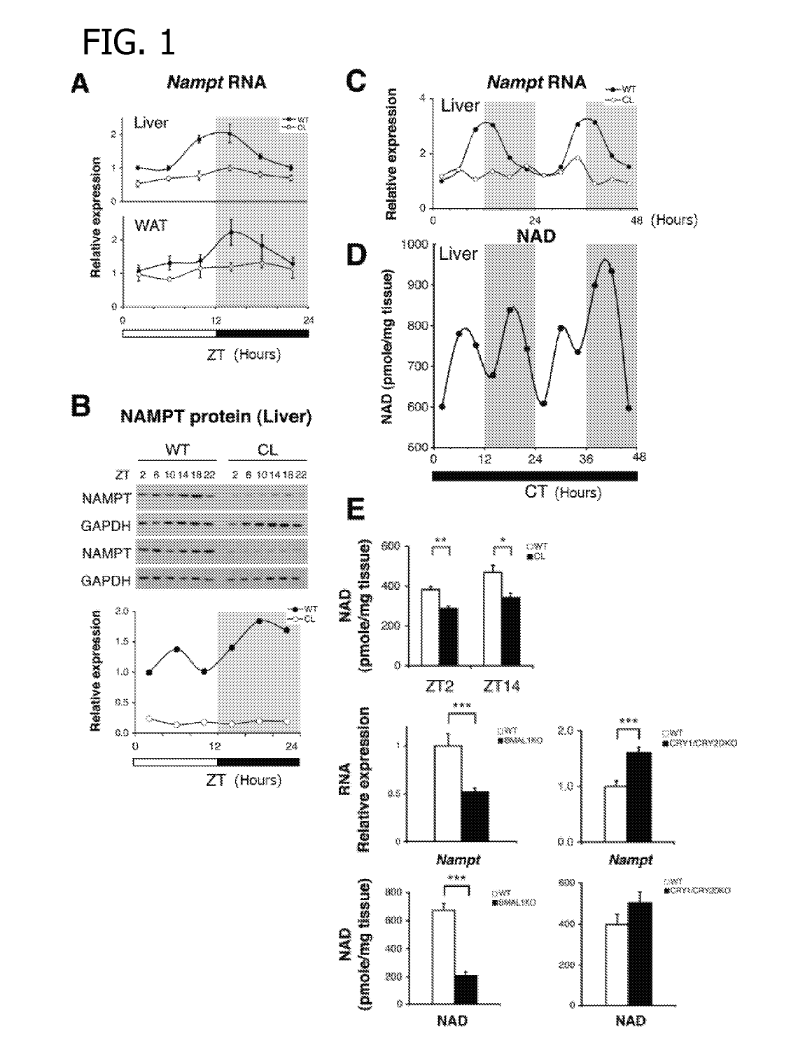

Results. Expression levels of Nampt RNA displayed a robust diurnal pattern in both wild-type mouse liver and white adipose tissue (WAT), peaking at the beginning of the dark period (zeitgeber time (ZT) 14) (FIG. 1A). Expression levels of NAMPT protein also showed a diurnal pattern of oscillation across the 24 hr light-dark cycle in liver (FIG. 1B), with a reduction in NAMPT protein levels prior to the onset of the dark period (FIG. 1B). Nampt RNA oscillation is circadian in nature, as a robust oscillation of Nampt RNA for 48 hr in liver isolated from wild-type mice that had been maintained in constant darkness (p<0.01, one-way ANOVA) (FIG. 1C) was found. Furthermore, the levels of Nampt RNA and protein were lower across the entire light-dark cycle in liver and WAT of Clock^{Δ19} mutant mice, and the diurnal oscillation of Nampt RNA and protein was abolished in Clock^{Δ19} mutant mice (FIGS. 1A and 1B). The robust Nampt RNA oscillation during constant darkness was also abolished completely in Clock^{Δ19} mutant mouse liver (FIG. 1C), indicating that the core clock machinery is required for the circadian control of NAMPT expression.

Because NAMPT is the rate-limiting enzyme in the predominant NAD⁺ biosynthetic pathway in mammals, an experiment was conducted to determine whether tissue NAD⁺ levels also display a circadian oscillation pattern in wild-type liver across 48 hrs from mice maintained in constant darkness. Liver NAD⁺ levels showed a very similar bimodal circadian oscillation pattern to that of NAMPT protein, and the lowest levels of NAD⁺ occurred with a rhythmicity of 24 hrs (FIG. 1D). A decrease in NAD⁺ levels was also observed around CT10-14 and again 24 hrs later (CT34), generating the observed bimodal oscillation pattern (FIG. 1D). The degree to which NAD⁺ levels increased from baseline (40-53%) during these daily cycles, and the concentrations of NAD⁺ that we measured, fall within the physiological range of reported alterations in NAD⁺ levels (J. T. Rodgers et al., Nature 434, 113 (2005); D. Chen et al., Genes Dev. 22, 1753 (2008)).

A bimodal oscillation of the NAMPT protein (FIG. 1B) and the metabolite NAD⁺ (FIG. 1D) was observed, suggesting that NAMPT protein levels might be regulated at the posttranslational level in response to changes in activity or ingestive behavior.

NAD⁺ levels were significantly reduced in Clock^{Δ19} mutant liver during both the light (ZT2) and dark (ZT14) periods (FIG. 1E). Furthermore, it was observed that mice deficient in Bmal1 (Bmal1^{-/-}) (M. K. Bunger et al., Cell 103, 1009 (2000)), the heterodimeric binding partner of CLOCK, also exhibited a significant reduction in both Nampt RNA and NAD⁺ levels in liver (FIG. 1E). Finally, mice deficient for both CRY1 and CRY2 (M. H. Vitaterna et al., Proc. Natl. Acad. Sci. USA 96, 12114 (1999)), displayed a significant increase in Nampt RNA levels and a corresponding increase in NAD⁺ levels in liver, which is consistent with Nampt being a target gene of CLOCK:BMAL1 (FIG. 1E). The rhythmic oscillation in RNA and protein levels of NAMPT thus leads to a circadian oscillation of NAD⁺ levels in the living animal.

Because NAD⁺ levels do not usually change to a large degree and 20-70% changes from basal levels are enough to convey a variety of biological effects in vivo and in vitro (J. R. Revollo, A. A. Grimm, S. Imai, J. Biol. Chem. 279, 50754 (2004); J. T. Rodgers et al., Nature 434, 113 (2005); D. Chen et al., Genes Dev. 22, 1753 (2008); H. Yang et al., Cell 130, 1095 (2007)), the observed scale of changes in this NAD+oscillation are sufficient to mediate important physiological effects on metabolic functions in the liver. Variation in NAD is likely also influenced by age, as plasma NMN levels are found to decline as mice age (K. M. Ramsey, K. F. Mills, A. Satoh, S. Imai, Aging Cell 7, 78 (2008)).

### Example 2

The peak in NAMPT and NAD⁺ in liver of wild-type mice (FIGS. 1B, 1D, and 1E) is consistent with a previous report that endogenous SIRT1, an NAD⁺-dependent and nutrient-responsive deacetylase, displays a diurnal oscillation in activity with a peak around ZT15 (Y. Nakahata et al., Cell 134, 329 (2008)). An experiment was conducted to examine whether SIRT1 activity is affected by altering NAMPT-mediated NAD⁺ biosynthesis in primary hepatocytes using the specific chemical inhibitor of NAMPT, FK866 (M. Hasmann, I. Schemainda, Cancer Res. 63, 7436 (2003)).

Luciferase Assays. HEK293 and HepG2 cells were transiently transfected with Clock (100 ng), Bmal1 (100 ng), firefly luciferase reporter (100 ng), and renilla luciferase reporter (control for transfection efficiency) (4 ng) using Lipofectamine 2000. Increasing doses of Sirt1 (200-800 ng), resveratrol (20, 50, 100 uM), nicotinamide (1, 5, 10 mM), Ex-527 (0.1 or 1.0 uM), or FK866 (10 nM) were added as indicated. 48 hrs following transfection, cells were lysed and luciferase activity monitored using the Dual-Luciferase® Reporter Assay System (Promega). Firefly luciferase values were normalized to renilla luciferase values, and a minimum of 3 independent experiments was performed in triplicate for each assay.

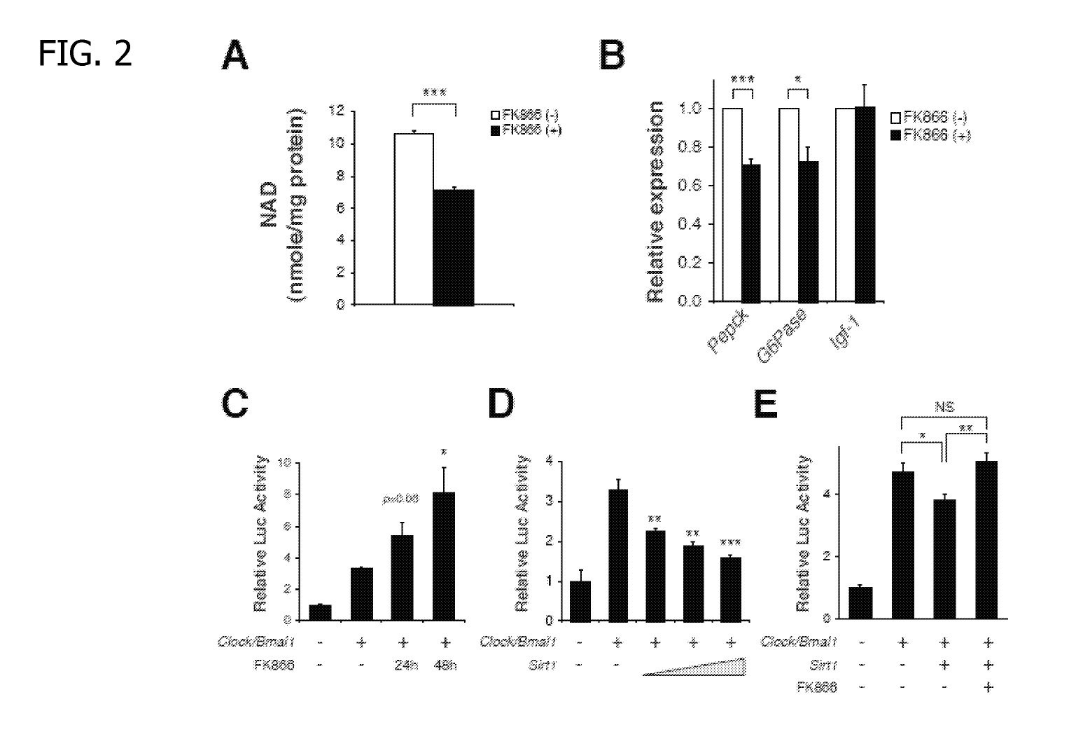

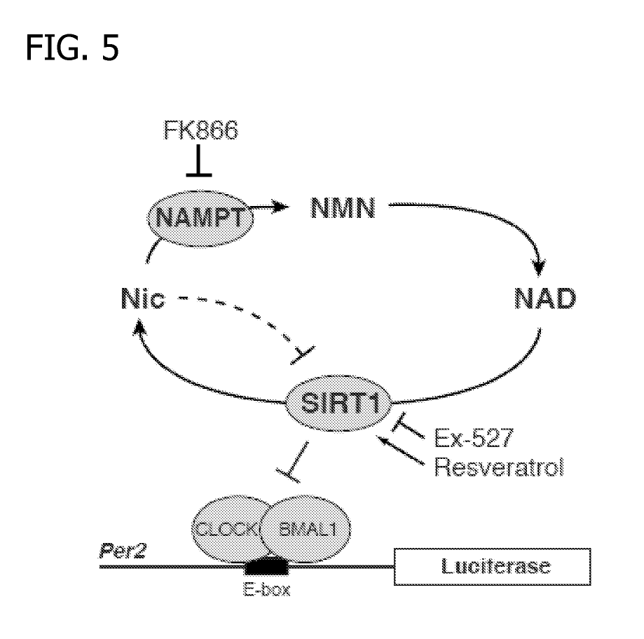

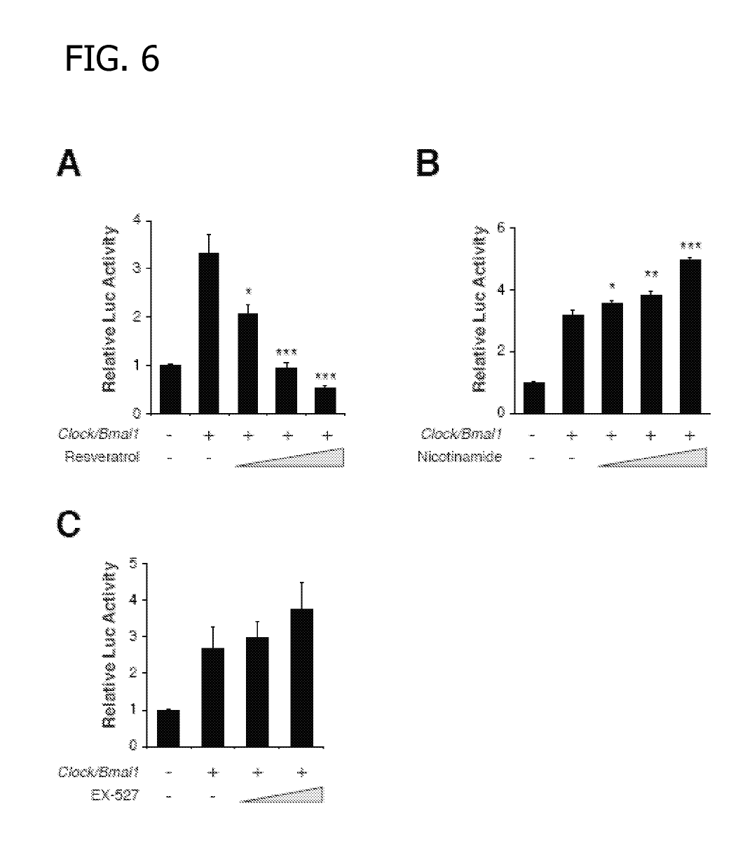

Results. FK866 significantly inhibited NAD⁺ biosynthesis (FIG. 2A), resulting in ˜30% reduction in mRNA expression levels of two major SIRT1 target genes in the liver, Pepck (phosphoenolpyruvate carboxykinase) and G6 Pase (glucose-6-phosphatase) (J. T. Rodgers et al., Nature 434, 113 (2005)), but not an unrelated gene, Igf-1 (insulin-like growth factor-1) (FIG. 2B). Using Per2:luciferase transcriptional reporter assays in HEK293 cells (FIGS. 2C, 2D, 2E, and 5), inhibition of NAMPT by FK866 was shown to lead to a significant increase in the CLOCK:BMAL1-driven transcription of the Per2:luciferase reporter (FIG. 2C), indicating that reduced NAMPT-mediated NAD⁺ biosynthesis released CLOCK:BMAL1 from the SIRT1-dependent suppression. Consistent with this notion, SIRT1 suppressed transcription of Per2:luciferase in a dose-dependent manner (FIG. 2D). Additionally, resveratrol, a putative SIRT1 activator, also inhibited Per2:luciferase transcription (FIGS. 5 and 6A). In contrast, inhibition of SIRT1 by high doses of nicotinamide activated Per2:luciferase transcription, and the selective SIRT1 inhibitor EX-527 showed a similar trend (FIGS. 5, 6B, and 6C). Furthermore, FK866 abrogated the SIRT1-dependent suppression of CLOCK:BMAL1-mediated transcription (FIG. 2E).

### Example 3

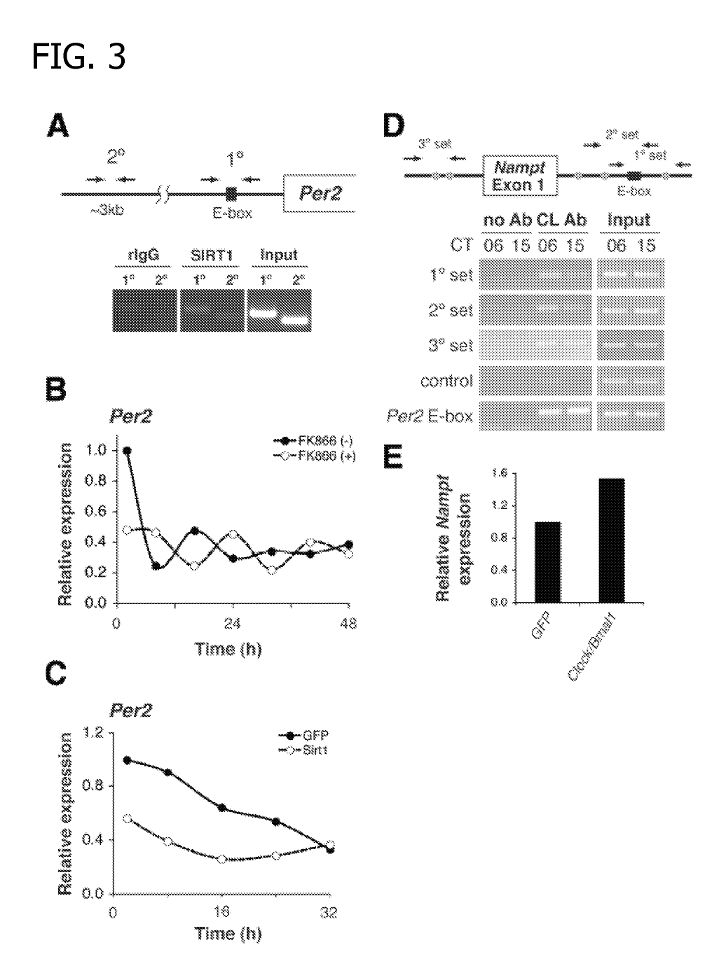

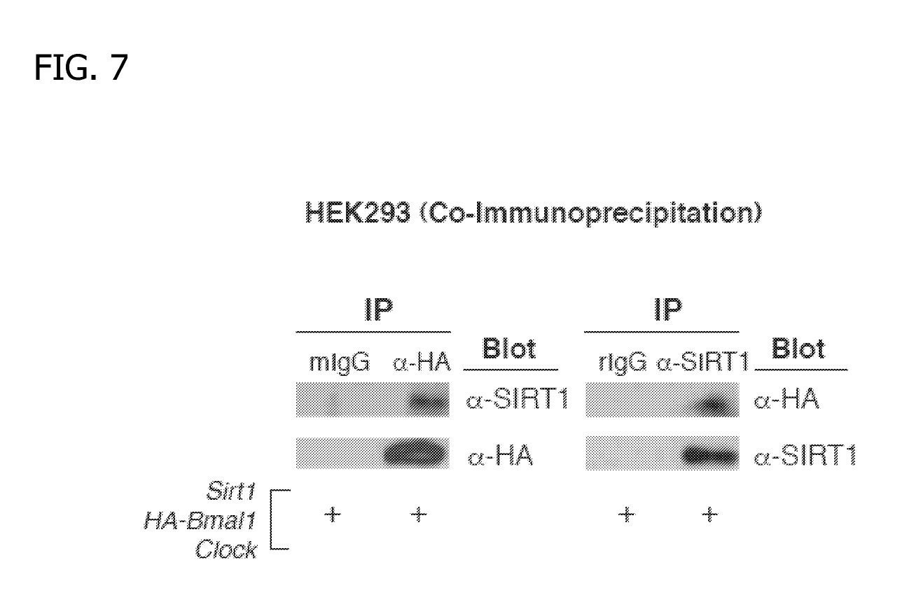

To investigate the effect of this regulatory pathway on clock gene expression, it was first confirmed that SIRT1 binds to BMAL1 (FIG. 7) (Y. Nakahata et al., Cell 134, 329 (2008); G. Asher et al., Cell 134, 317 (2008)), and it was then determined that SIRT1 localizes to the E-box of the Per2 promoter (FIG. 3A), suggesting that Per2 is a target of the NAMPT/NAD⁺-driven pathway. The effect of FK866 on Per2 rhythms in primary hepatocytes was then examined.

Primary Hepatocyte Isolation and Adenoviral Infection. Primary hepatocytes were isolated from mice by hepatic portal collagenase perfusion as previously described (J. E. Klaunig et al., In Vitro 17, 913 (1981)). Six hours following isolation, primary hepatocytes were infected with either SIRT1- or green fluorescent protein (GFP)— expressing adenovirus. Infected hepatocytes were cultured overnight in DMEM media containing 10% FBS and penicillin/streptomycin and then synchronized as described below.

Synchronization of Primary Hepatocytes by Serum Shock. Hepatocytes were synchronized by changing to media containing 50% horse serum (t=0) for 2 hrs and then maintaining cells in media containing 0.5% FBS (A. Balsalobre, F. Damiola, U. Schibler, Cell 93, 929 (1998)). For FK866 application, 200 nM FK866 was added to the culture at t=2. RNA samples were collected at the indicated time points, and the expression of clock genes was examined by quantitative real-time PCR as described above. Damping rate was assessed as the decay rate of the oscillations of averaged Per2 RNA abundance assuming exponential decay.

Adenoviral Infection in Mouse Embryonic Fibroblasts (MEFs). CLOCK/BMAL1 and GFP were overexpressed in primary mouse embryonic fibroblasts (MEFs) using an adenoviral vector as previously described (T. C. He et al., Proc. Natl. Acad. Sci. USA 95, 2509 (1998)). To restrict the effects of CLOCK/BMAL1 overexpression to first-order genes regulated by CLOCK/BMAL1, cycloheximide was added at 2 mg/ml following the adenoviral infection in order to block translation from the RNA regulated by the overexpressed transcription factors. Nampt mRNA levels were measured by quantitative real-time PCR as described above.

Results. Serum shock induced a Per2 oscillation in vehicle-treated hepatocytes that damped rapidly after one cycle (2.2 day damping rate), whereas FK866 treatment prolonged the oscillation of Per2 (5.3 day damping rate) (FIG. 3B). Reduction in NAMPT-mediated NAD⁺ biosynthesis may promote a more robust oscillation of clock target gene expression by releasing CLOCK:BMAL1 from SIRT1-mediated suppression. Consistent with this notion, adenoviral Sirt1 transduction of primary hepatocytes suppressed the expression and the oscillation of Per2, although adenoviral infection itself altered the Per2 oscillation pattern (FIG. 3C). Therefore, the NAMPT/NAD⁺-driven pathway modulates circadian transcription patterns in mammals.

### Example 4

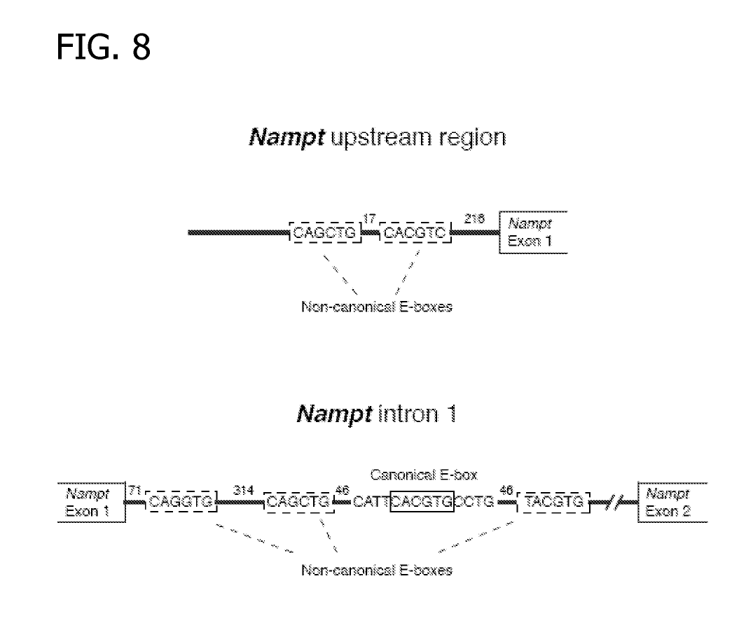

To determine if this pathway comprises a feedback loop involving regulation of the Nampt gene by CLOCK:BMAL1, chromatin immunoprecipitation (ChIP) was performed to test whether the CLOCK:BMAL1 complex binds to canonical or non-canonical E-box motifs in the promoter and first intron of the Nampt gene (FIG. 8).

Chromatin Immunoprecipitation. For primary hepatocytes, crosslinking and nuclear preparation were performed as adapted from the Misonix ChIP protocol, 2005. Sonication sheared the DNA to an average size of ˜400 base pairs. For liver, cross-linked nuclei were prepared from liver collected at CT6 and 15 as previously described in Lee et al., 2001 (C. Lee, J. P. Etchegaray, F. R. Cagampang, A. S. Loudon, S. M. Reppert, Cell 107, 855 (2001)). Immunoprecipitation was then performed with either an α-SIRT1 antibody (AS-16, Sigma), an α-CLOCK antibody (C. Lee, J. P. Etchegaray, F. R. Cagampang, A. S. Loudon, S. M. Reppert, Cell 107, 855 (2001)), or appropriate IgG controls. Crosslinks were reversed, and immunoprecipitated DNA was precipitated and purified. Standard polymerase chain reaction (PCR) amplification was performed to amplify Per2 and Nampt promoter regions.

Co-Immunoprecipitation. HEK293 and HepG2 cells were transfected using Lipofectamine 2000 (Invitrogen) with mouse Sirt1, Clock, and HA-Bmal1 DNA in CMV expression vectors. After 48 hrs, the cells were lysed, precleared with either mouse or rabbit IgG and Protein A/G or Protein A Plus agarose bead slurry (Pierce Biotechnology), respectively, and 500 μg of lysates were incubated overnight at 4° C. with either α-SIR2 (Millipore) or α-HA (Cell Signaling Technology) primary antibodies. The following day, lysates were incubated with the appropriate agarose bead slurry, followed by washes and then resuspension in sample buffer. Samples were briefly vortexed, heated, and subjected to SDS-PAGE analysis and Western blotting.

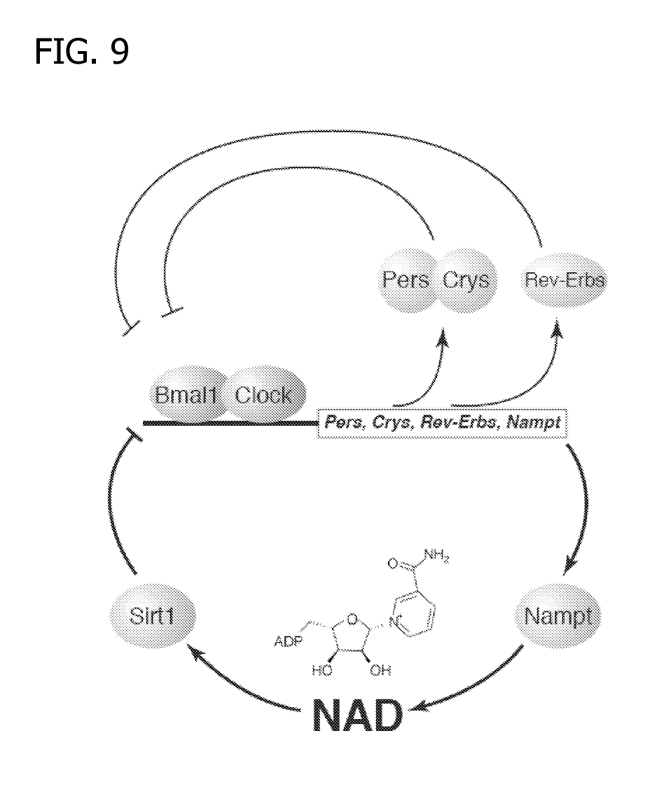

Results. ChIP detected CLOCK in these regions (FIG. 3D, first, second, and third panels), but not in a non-specific upstream region (FIG. 3D, fourth panel). A stronger signal for CLOCK at CT6 compared to CT15 (FIG. 3D) was observed, suggesting rhythmic binding of CLOCK:BMAL1 in a promoter context-dependent manner (C. Lee, J. P. Etchegaray, F. R. Cagampang, A. S. Loudon, S. M. Reppert, Cell 107, 855 (2001); J. A. Ripperger, U. Schibler, Nat. Genet. 38, 369 (2006)). Transduction of Clock/Bmal1 into mouse embryonic fibroblasts appeared to up-regulate Nampt expression ˜1.6-fold (FIG. 3E). Together, these data demonstrate that NAMPT-mediated NAD⁺ biosynthesis comprises a feedback loop in which NAD⁺ functions as a metabolic oscillator and regulates the core clock machinery through SIRT1 (FIG. 9).

1. A method of regulating a biological function in a mammal in need thereof, said function being affected by the circadian clock, the method comprising administering to the mammal a compound selected from nicotinamide or salts or prodrugs thereof, nicotinamide mononucleotide (NMN) or salts or prodrugs thereof, nicotinamide adenine dinucleotide (NAD) or salts or prodrugs thereof, nicotinamide phosphoribosyltransferase (NAMPT), or combinations thereof.

2. A method of modulating sleep in a mammal in need thereof, said method comprising administering to the mammal a compound selected from nicotinamide or salts or prodrugs thereof, nicotinamide mononucleotide (NMN) or salts or prodrugs thereof, nicotinamide adenine dinucleotide (NAD) or salts or prodrugs thereof, nicotinamide phosphoribosyltransferase (NAMPT), or combinations thereof.

3. A method of treating or preventing a disorder in a mammal mediated by the function of the circadian clock, said method comprising administering to the mammal a compound selected from nicotinamide or salts or prodrugs thereof, nicotinamide mononucleotide (NMN) or salts or prodrugs thereof, nicotinamide adenine dinucleotide (NAD) or salts or prodrugs thereof, nicotinamide phosphoribosyltransferase (NAMPT), or combinations thereof.

4. The method of claim 1, wherein the compound is nicotinamide or salts or prodrugs thereof, nicotinamide mononucleotide (NMN), nicotinamide adenine dinucleotide (NAD), nicotinamide phosphoribosyltransferase (NAMPT), or combinations thereof.

5. The method of claim 1, wherein the biological function is metabolic activity of the mammal.

6. The method of claim 5, wherein administration of the compound increases metabolic activity.

7. The method of claim 5, wherein administration of the compound decreases metabolic activity.

8. The method of claim 1, wherein the compound is nicotinamide phosphoribosyltransferase (NAMPT).

9. The method of claim 1, wherein the compound is a combination of nicotinamide mononucleotide (NMN) and nicotinamide phosphoribosyltransferase (NAMPT).

10. The method of claim 8, wherein the nicotinamide phosphoribosyltransferase (NAMPT) has a peptide sequence selected from the group consisting of:
(a) SEQ ID NO: 1;
(b) SEQ ID NO: 2;
(c) a polypeptide capable of catalyzing the conversion of nicotinamide to nicotinamide mononucleotide (NMN), an amino acid sequence of the polypeptide comprising the amino acid sequence of SEQ ID NO: 1;
(d) a polypeptide capable of catalyzing the conversion of nicotinamide to nicotinamide mononucleotide (NMN), an amino acid sequence of the polypeptide comprising the amino acid sequence of SEQ ID NO: 2;
(e) a polypeptide capable of catalyzing the conversion of nicotinamide to nicotinamide mononucleotide (NMN), the polypeptide having an amino acid sequence with at least about 90% homology to SEQ ID NO: 1 and conservative amino acid substitutions; and
(f) a polypeptide capable of catalyzing the conversion of nicotinamide to nicotinamide mononucleotide (NMN), the polypeptide having an amino acid sequence with at least about 90% homology to SEQ ID NO: 2 and conservative amino acid substitutions.

11. The method of claim 8, wherein the NAMPT is a secreted form of NAMPT.

12. The method of claim 1, wherein the compound is nicotinamide mononucleotide (NMN).

13. The method of claim 5, wherein the metabolic activity is increasing the anabolic activity of the liver.

14. The method of claim 5, wherein the metabolic activity is increasing the catabolic activity of the liver.

15. The method of claim 3, wherein the disorder is caused by dysfunction of the circadian clock.

16. The method of claim 15, wherein the disorder is improper cycling between feeding and fasting cycles, hyperglycemia, and hypoglycemia.

17. The method of claim 2, wherein the sleep modulation treats a sleep disorder.

18. The method of claim 3, wherein the disorder is a sleep disorder.

19. The method of claim 18, wherein the sleep disorder is selected from the group consisting of insomnia, advanced sleep phase syndrome, delayed sleep phase syndrome, inconsistent sleep/wake cycles, and narcolepsy.

20. The method of claim 3, wherein the disorder is caused by travel to or across one or more time zones (jet lag), by a shift into or out of daylight savings time, by a change in work shifts, by night shift work, or by medication.
